(19) United States
(12) Patent Application Publication
(10) Pub. No.: US 2016/0332935 A1
(43) Pub. Date: Nov. 17, 2016

(54) Integrated Process for Making HCFO-1233zd and HFC-245fa

(71) Applicant: Honeywell International Inc., Morris Plains, NJ (US)

(72) Inventors: Stephen A. Cottrell, Baton Rouge, LA (US); Gustavo Cerri, Parsippany, NJ (US); Yuon Chiu, Denville, NJ (US); Jennifer W. Mcclaine, Branchburg, NJ (US)

(21) Appl. No.: 15/088,709

(22) Filed: Apr. 1, 2016

Related U.S. Application Data
(60) Provisional application No. 62/160,026, filed on May 12, 2015.

(51) Int. Cl. C07C 17/20

(52) U.S. Cl. CPC C07C 17/20

(57) ABSTRACT

A process is described wherein otherwise unusable by-products from a process for the manufacture of trans HCFO-1233zd(E) are converted to a valuable product by introducing them into a process for the production of HFC-245fa. The process includes the catalytic hydrofluorination of a reaction mixture comprising the HCFO-1233zd production by-products.

### CROSS REFERENCE TO RELATED APPLICATIONS

This application claims domestic priority to commonly owned copending U.S. Provisional Application Ser. No. 62/160,026, filed May 12, 2015, the disclosure of which is hereby incorporated herein by reference.

### BACKGROUND OF THE INVENTION

U.S. Pat. No. 9,045,386 describes a process to produce trans-1-chloro-3,3,3-trifluoropropene (HCFO-1233zd(E)) at high purity on a commercial scale. This patent is hereby incorporated herein by reference.

It has been discovered that certain by-products can be generated in the HCFO-1233zd(E) manufacturing process, including HCFC-241 isomers, HCFC-242 isomers, HCFC-243 isomers, HCFC-244 isomers, and the cis-isomer of HCFO-1233zd. These by-products are produced at a ratio of 0.25-35 kg per kg of the trans-isomer of HCFO-1233zd. Since these by-products are not simple precursors to HCFO-1233zd, they cannot be readily recycled in the process. The volume of these by-products and their cost of disposal could significantly impact the economic viability of this commercial process.

### SUMMARY OF THE INVENTION

This invention is based on the discovery that the HCFO-1233zd by-products can instead be used in a manufacturing process for the production of 1,1,1,3,3-pentafluoro-propane (HFC-245fa), another commercially useful product. Other sources of the isomers of HCFC-241, HCFC-242, HCFC-243, and the cis isomer of HCFO-1233zd, may likewise be used in this process—since these materials may be available from processes that are not based on them only being HCFO-1233zd by-products.

In one embodiment, combining these two manufacturing processes into an integrated manufacturing scheme is accomplished by feeding the isolated by-products from the 1233zd process to a reactor used to produce HFC-245fa, either alone, or in tandem with the normal HCC-240fa raw materials and HF. The HCFO-1233zd by-products are then converted into HFC-245fa, and are recovered therefrom as a commercially viable product.

The ability to integrate the HCFO-1233zd process with a HFC-245fa process removes the financial penalty of producing un-recyclable by-products and greatly improves the commercial viability of the HCFO-1233zd production process.

It should be appreciated by those persons having ordinary skill in the art(s) to which the present invention relates that any of the features described herein in respect of any particular embodiment and/or embodiment of the present invention can be combined with one or more of any of the other features of any other embodiments and/or embodiments of the present invention described herein, with modifications as appropriate to ensure compatibility of the combinations. Such combinations are considered to be part of the present invention contemplated by this disclosure.

It is to be understood that both the foregoing general description and the following detailed description are exemplary and explanatory only and are not restrictive of the invention as claimed. Other embodiments will be apparent to those skilled in the art from consideration of the specification and practice of the invention disclosed herein.

### DETAILED DESCRIPTION OF THE INVENTION

The present invention was based on the realization that the process for producing HCFO-1233zd and the process for producing HFC-245fa both utilize similar raw materials. The present inventors thus theorized that the HCFO-1233zd process by-products could be used as precursors in the production of HFC-245fa.

U.S. Pat. Nos. 5,574,192 and 5,616,819 describe processes for the production of HFC-245fa from HCC-240fa. However these patents do not teach or suggest the use of the 1233 by-products as raw materials for HFC-245fa production. These patents are hereby incorporated herein by reference.

Fluorination catalysts useful in the process of the invention include: (I) pentavalent antimony, niobium, arsenic and tantalum halides; (II) pentavalent antimony, niobium, arsenic and tantalum mixed halides; and (III) mixtures of pentavalent antimony, niobium, arsenic and tantalum halide catalysts. Examples of catalysts of group (I) include antimony pentachloride and antimony pentafluoride. Examples of catalysts of group (II) include SbCl₂F₃ and SbBr₂F₃. Examples of catalysts of group (III) include a mixture of antimony pentachloride and antimony pentafluoride.

Pentavalent antimony, niobium, arsenic and tantalum halides are commercially available, and mixed halides thereof are created in situ upon reaction with HF. Antimony pentachloride is preferred because of its low cost and availability. Pentavalent antimony mixed halides of the formula SbClₙF₅₋ₙ where n is 0 to 5 are more preferred. The fluorination catalysts used in this invention preferably have a purity of at least about 97%. Although the amount of fluorination catalyst used may vary widely, we recommend using from about 5 to about 50%, or preferably from about 10 to about 25% by weight catalyst relative to the organics.

The temperature at which the fluorination reaction is conducted and the period of reaction will depend on the starting material and catalyst used. One of ordinary skill in the art can readily optimize the conditions of the reaction without undue experimentation to get the claimed results, but the temperature will generally be in the range of from about 50° to about 175° C., and preferably from about 115° C. to about 155° C., for a period of, for example, from about 1 to about 25 hours, and preferably from about 2 to about 8 hours.

Pressure is not critical. Convenient operating pressures range from about 1500 to about 5000 KPa, and preferably from about 1500 to about 2500 KPa.

The equipment in which the fluorination reaction is conducted is preferably made of corrosion resistant material such as Inconel or Monel.

HFC-245fa may be recovered from the mixture of unreacted starting materials, by-products, and catalyst by any means known in the art, such as distillation and extraction. At the end of the heating period, i.e., the amount of time for complete reaction in batch mode operations, the fluorination reaction product and remaining HF may be vented through a valve on the autoclave head, which in turn is connected to an acid scrubber and cold traps to collect the product. Alternatively, unreacted HF and organics may be vented and condensed, and the HF layer recycled to the reactor. The organic layer can then be treated, i.e., washed with an aqueous base, to remove dissolved HF and distilled. This isolation procedure is particularly useful for a continuous fluorination process.

### EXAMPLES

The following examples illustrate the advantages of this invention but are not to be construed as limiting the invention.

### Example 1

30,000 lbs of mixture of isomers of HCFC-241, HCFC-242, HCFC-243, and the cis isomer of HCFO-1233zd was fed to a commercial reactor producing HFC-245fa from HCC-240fa. The reaction was conducted at a temperature of 215° F. and a pressure of 150 psig. Reaction products were removed continuously. HFC-245fa meeting all product specifications was produced from the mixture.

Example 2

In a laboratory setting, a small quantity of a mixture of isomers of HCFC-241, HCFC-242, HCFC-243, and the cis isomer of HCFO-1233zd was mixed with HF and fluorinated antimony pentachloride catalyst. The reactions were run by first charging SbCl₅ and HF at room temperature with agitation of about 180 RPM. The HCl generated by the fluorination of the catalyst was vented to a scrubber carboy containing KOH solution. The reactor was then heated to 95° C. while the organic feed cylinder was also heated to about 95° C. When at temperature the organic was quickly charged.

It was observed for each experiment that the temperature first decreased by about 10° C. to 12° C. and then heated up to between 112° C. and 118° C. Thereafter, within a few minutes, the reaction cooled back down to about 95° C. The pressure of each reaction increased to between 700 to 800 psig. The pressure rise stopped after about 1-2 minutes for all of the runs. The reactions were held at the final temperature for between 5 to 8 additional minutes. Then the reaction was stopped abruptly by opening a vent valve to an evacuated liquid N₂ cooled 500 cc product collection cylinder (PCC).

The remaining reactor contents were quenched with about 100 grams of water and 20 grams of MeCl₂. HFC-245fa and normally-observed pre-cursors to HFC-245fa were the observed reaction products.

As used herein, the singular forms “a”, “an” and “the” include plural unless the context clearly dictates otherwise. Moreover, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

From the foregoing, it will be appreciated that although specific examples have been described herein for purposes of illustration, various modifications may be made without deviating from the spirit or scope of this disclosure. It is therefore intended that the foregoing detailed description be regarded as illustrative rather than limiting, and that it be understood that it is the following claims, including all equivalents, that are intended to particularly point out and distinctly claim the claimed subject matter.

What is claimed is:

1. A process for making 1,1,1,3,3-pentafluoropropane (HFC-245fa) comprising the catalytic hydrofluorination of a reaction mixture comprising by-products formed during the production of 1-chloro-3,3,3-trifluoropropene (HCFO-1233zd), wherein the byproducts are selected from the group consisting of isomers of HCFC-241, HCFC-242, HCFC-243 and mixtures thereof

2. The process of claim 1, wherein the HCFO-1233zd by-products are isolated from a process for the production of trans-1-chloro-3,3,3-trifluoropropene (HCFO-1233zd(E)) and wherein the by-products further comprise the cis isomer of HCFO-1233zd.

3. The process of claim 1, wherein the reaction mixture further includes HCC-240fa.

4. The process of claim 1, wherein the catalyst is selected from the group consisting of (I) pentavalent antimony, niobium, arsenic and tantalum halides; (II) pentavalent antimony, niobium, arsenic and tantalum mixed halides; and (III) mixtures of pentavalent antimony, niobium, arsenic and tantalum halide catalysts.

5. The process of claim 4, wherein the catalyst is selected from the group consisting of antimony pentachloride and antimony pentafluoride.

6. The process of claim 4, wherein the catalyst is selected from the group consisting of SbCl₂F₃ and SbBr₂F₃.

7. The process of claim 4, wherein the catalyst is a mixture of antimony pentachloride and antimony pentafluoride.

8. The process of claim 4, wherein the catalyst is fluorinated antimony pentachloride.

9. The process of claim 1, wherein the catalyst is fluorinated antimony pentafluoride.

10. A process for making 1,1,1,3,3-pentafluoropropane (HFC-245fa) comprising the catalytic hydrofluorination of a reaction mixture comprising isomers of HCFC-241, HCFC-242, HCFC-243, and the cis isomer of HCFO-1233zd, isolated from a process for the production of trans-1-chloro-3,3,3-trifluoropropene (HCFO-1233zd(E)).

11. The process of claim 10, wherein the reaction mixture further includes HCC-240fa.

12. The process of claim 10, wherein the catalyst is selected from the group consisting of (I) pentavalent antimony, niobium, arsenic and tantalum halides; (II) pentavalent antimony, niobium, arsenic and tantalum mixed halides; and (III) mixtures of pentavalent antimony, niobium, arsenic and tantalum halide catalysts.

13. The process of claim 12, wherein the catalyst is selected from the group consisting of antimony pentachloride and antimony pentafluoride.

14. The process of claim 12, wherein the catalyst is selected from the group consisting of SbCl₂F₃ and SbBr₂F₃.

15. The process of claim 12, wherein the catalyst is a mixture of antimony pentachloride and antimony pentafluoride.

16. The process of claim 10, wherein the catalyst is fluorinated antimony pentachloride.

17. The process of claim 10, wherein the catalyst is fluorinated antimony pentafluoride.

18. A process for making 1,1,1,3,3-pentafluoropropane (HFC-245fa) comprising the catalytic hydrofluorination of a reaction feed selected from the group consisting of isomers of HCFC-241, HCFC-242, HCFC-243, the cis isomer of HCFO-1233zd, and mixtures thereof.
